(19) United States
(12) Patent Application Publication
(10) Pub. No.: US 2005/0049299 A1
(43) Pub. Date: Mar. 3, 2005

(54) Selective inhibitors of stat-3 activation and uses thereof

(76) Inventor: Bharat B. Aggarwal, Houston, TX (US)

Correspondence Address: Benjamin Aaron Adler;ADLER & ASSOCIATES, 8011 Candle Lane, Houston, TX, 77071, US

(21) Appl. No.: 10/925,814

(22) Filed: Aug. 25, 2004

Related U.S. Application Data
(60) Provisional application No. 60/497,842, filed on Aug. 26, 2003.

(51) Int. Cl. A61K 31/12

(52) U.S. Cl. 514/456; 514/689

(57) ABSTRACT

The present invention provides a method of treating a cancerous or pre-cancerous state in an individual in need of such treatment, comprising the step of administering a pharmacologically effective dose of a curcuminoid to the individual.

### CROSS-REFERENCE TO RELATED APPLICATION

This patent application claims benefit of priority of provisional patent application U.S. Ser. No. 60/497,842, filed Aug. 26, 2003, now abandoned.

### BACKGROUND OF THE INVENTION

1. Field of the Invention

The present invention relates generally to the molecular biology of stat-3. More specifically, the present invention relates to compounds that can selectively inhibit stat-3 activation induced by various inflammatory stimuli and other apoptotic stimuli.

2. Description of the Related Art

Multiple myeloma (MM) is a B cell malignancy characterized by the latent accumulation in bone marrow of secretory plasma cells with a low proliferative index and an extended life span (1). Multiple myeloma accounts for 1% of all cancers and >10% of all hematologic cancers. Agents used to treat myeloma includes combinations of vincristine, BCNU, melphalan, cyclophosphamide, adriamycin, and prednisone or dexamethasone (2). Usually, patients younger than 65 years are treated with high-dose melphalan with autologous stem-cell support, and older patients who cannot tolerate such intensive treatment receive standard-dose oral melphalan and prednisone. Despite these treatments, only 5% of patients achieve complete remission and the median survival is only 30-36 months (3, 4).

The dysregulation of the apoptotic mechanism in plasma cells is considered a major underlying factor in the pathogenesis and subsequent chemoresistance in multiple myeloma. It is established that IL-6, produced in either an autocrine or paracrine manner, has an essential role in the malignant progression of multiple myeloma by regulating the growth and survival of tumor cells (5, 6). IL-6 induces intracellular signaling through a member of the signal transducers and activators of transcription (STAT) family. Engagement of cell surface cytokine receptors activates the Janus kinase (JAK) family of protein tyrosine kinases, which phosphorylate and activate cytoplasmic STAT proteins (7, 8). Activated STATs dimerize and translocate to the nucleus, where they bind to specific DNA response elements and induce expression of STAT-regulated gene expression.

One STAT family member, STAT3, has been described in mediating the IL-6 signaling through interaction with the IL-6 receptor, and studies using dominant-negative STAT3 proteins have demonstrated a requirement of STAT3 signaling in tumor transformation (9, 10). Evidence is accumulating that constitutive activation of STAT3 proteins occurs frequently in human tumor cells (11-14), implicating aberrant STAT3 signaling as an important process in malignant progression.

Recently, Catlett-Falcone et al. have shown that human multiple myeloma cells also express constitutively activated STAT3, which confers resistance to apoptosis in these cells through expression of high levels of the anti-apoptotic protein Bcl-x_{L} (15-17). Bcl-2 over-expression, another important characteristic of most multiple myeloma cell lines (18), rescues these tumor cells from chemotherapy-induced apoptosis (4, 19). Thus pharmacologically safe and effective agents that can block constitutive or inducible activation of STAT3 would be useful treatments for multiple myeloma and other diseases.

The prior art is deficient in pharmacologically safe and effective agents that can block constitutive as well as inducible activation of STAT3 as treatments have a potential for multiple myeloma and other diseases. The present invention fulfills this long-standing need and desire in the art.

### SUMMARY OF THE INVENTION

Numerous reports suggest that interleukin-6 (IL-6) promotes survival and proliferation of multiple myeloma (MM) cells through the phosphorylation of a cell signaling protein, STAT3. Thus agents that suppress STAT3 phosphorylation have potential for the treatment of multiple myeloma. The present invention demonstrates that curcumin (diferuloylmethane), a pharmacologically safe agent in humans, inhibited IL-6-induced STAT3 phosphorylation and consequent STAT3 nuclear translocation. Curcumin had no effect on STAT5 phosphorylation but inhibited the interferon-a-induced STAT1 phosphorylation. The constitutive phosphorylation of STAT3 found in certain multiple myeloma cells was also abrogated by treatment with curcumin. Curcumin-induced inhibition of STAT3 phosphorylation was reversible. Compared with AG490, a well-characterized JAK2 inhibitor, curcumin was more rapid (30 min vs 8 h) and more potent (10 μM vs 100 μM) inhibitor of STAT3 phosphorylation. Similarly, dose of curcumin which completely suppressed proliferation of multiple myeloma cells, same dose of AG490 had no effect. In contrast, a cell permeable STAT3 inhibitor peptide that can inhibit the STAT3 phosphorylation mediated by Src blocked the constitutive phosphorylation of STAT3 and also suppressed the growth myeloma cells. TNF-α and lymphotoxin (LT) also induced the proliferation of multiple myeloma cells, but through a mechanism independent of STAT3 phosphorylation. In addition, dexamethasone-resistant multiple myeloma cells were found to be sensitive to curcumin. Overall, these results demonstrated that curcumin was a potent inhibitor of STAT3 phosphorylation and thus plays a role in the suppression of proliferation of multiple myeloma.

Additionally, the present invention demonstrates that IL-6 induces proliferation of human head and neck squamous cell carcinoma (HNSCC) cells. This effect of IL-6 was examined on MDA 1986LN, JMAR and MDA 686LN cells. Further, it also demonstrates that STAT3 is phosphorylated in all the head and neck squamous cell carcinoma cell lines except JMAR cells and that curcumin inhibited the phosphorylation of STAT3 in those cells. An exposure to curcumin (50 μM) for 1 hr completely inhibited STAT3 phosphorylation. However, STAT3 was not affected by curcumin. The present invention also demonstrates that curcumin prevented the translocation of STAT3 to nucleus of head and neck squamous cell carcinoma cells. Further, immunocytochemical analysis for phosphorylated STAT3 demonstrated that curcumin also inhibited STAT3 phosphorylation. The curcumin-induced inhibition of STAT3 phosphorylation in head and neck squamous cell carcinoma cells was reversible since removal of curcumin resulted in gradual increase in p-STAT3 levels. Additionally, the present invention also demonstrates that IL-6 induces STAT3 phosphorylation in JMAR cells, which was inhibited by curcumin. In comparison with AG490, curcumin was more potent inhibitor of STAT3 phosphorylation and cell proliferation of MDA 1986LN cells. Overall, these findings demonstrate that curcumin was a potent inhibitor of constitutively active and inducible STAT3 activation and could play an important role in inhibiting the proliferation of head and neck squamous cell carcinoma.

In one embodiment, the present invention provides a method of treating a cancerous or pre-cancerous state in an individual in need of such treatment, comprising the step of administering a pharmacologically effective dose of a curcuminoid to said individual.

In another embodiment, the present invention provides a method of reducing activated STAT3 expression in a cell, comprising the step of contacting said cell with pharmacologically effective dose of a curcuminoid or analogues thereof.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1A shows that curcumin inhibits constitutive STAT3 phosphorylation in U266 cells in dose- and time-dependent (FIG. 1B) manner. U266 cells (2×10⁶) were treated with the indicated concentrations of curcumin for 1 h, or treated with 50 μM curcumin for the indicated durations and levels of phosphorylated-STAT3 (upper panel) and STAT3 (lower panel) were examined in whole cell extracts by Western Blotting. FIG. 1C shows that curcumin specifically inhibits constitutive phosphorylation of STAT3 but not of STAT5 in U266 cells. U266 cells (2×10⁶) were treated with 50 μM curcumin for indicated durations, and examined for phosphorylated and nonphosphorylated-STAT3, and STAT5. FIG. 1D shows that curcumin inhibits IFN-a inducible STAT1 phosphorylation. U266 cells (2×10⁶) were preincubated with curcumin (50 μM) for indicated durations before treatment with IFN-α (10 ng/ml) for additional 30 min and then examined for phosphorylated and nonphosphorylated-STAT1.

FIG. 2 shows that curcumin induces redistribution of STAT3. U266 cells were incubated alone or with curcumin (50 μM) for 60 minutes and then analyzed for the distribution of STAT3 by immunocytochemistry (originial magnification, 200X).

FIGS. 3A-C show& that IL-6 induces STAT3 phosphorylation in human multiple myeloma RPMI 8226 cells and curcumin inhibits it. FIG. 3A shows that U266 but not other human multiple myeloma cells express phosphorylated STAT3. U266, RPMI 8226, MM.1S, MM.1R and OCl cells (2×10⁶) were lysed, and 30 μg of whole-cell extracts were resolved on 7.5% SDS-PAGE, electrotransferred to a nitrocellulose membrane, and probed for phosphorylated-STAT3 (upper panel) and STAT3 (lower panel). FIG. 3B shows that RPMI 8226 cells (2×10⁶ cells) were treated with IL-6 (10 ng/ml) for the indicated time and whole-cell extracts were prepared. FIG. 3C shows that RPMI 8226 cells (2×10⁶ cells) were treated with 50 μM curcumin for the indicated durations and stimulated with IL-6 (10 ng/ml) for 10 min and whole-cell extracts were prepared. Thirty micrograms of whole cell extracts were resolved on 7.5% SDS-PAGE, electrotransferred to a nitrocellulose membrane, and probed for the phosphorylated-STAT3 (upper panel) and stripped and reprobed for STAT3 (middle panel) and β-actin (lower panel).

FIG. 4 shows that curcumin-induced inhibition of STAT phosphorylation is reversible. U266 cells (2×10⁶ cells) were treated with 50 μM curcumin for the indicated durations (left panels) or treated for 1 h and washed with PBS two times to remove curcumin before resuspending in fresh medium. Cells were removed at indicated times and lysed to prepare the whole cell extract (right panels). Thirty micrograms of whole cell extracts were resolved on 7.5% SDS-PAGE, electrotransferred to a nitrocellulose membrane, probed for the phosphorylated-STAT3 (upper panels) and stripped and reprobed for STAT3 (middle panels) and β-actin (lower panels).

FIGS. 5A-C shows the effect of AG490 on STAT3 phosphorylation in U266 cells. FIG. 5A shows a structural similarity between curcumin and AG490. FIG. 5B shows that U266 cells (2×10⁶ cells) were treated with indicated concentrations of AG490 for 8 h, or as in FIG. 5C, treated with 100 μM AG490 for the indicated durations, and whole-cell extracts were prepared. Thirty micrograms of whole-cell extract was resolved on 7.5% SDS-PAGE, electrotransferred to a nitrocellulose membrane, probed for the phospho-STAT3 (upper panel), stripped, and reprobed for STAT3 (middle panel) and β-actin (lower panel).

FIGS. 6A-C show& the effect of cell permeable STAT3 inhibitor peptide (STAT3iP) on STAT3 phosphorylation and cell viability in U266 cells. FIG. 6A shows the sequence of STAT3iP. FIG. 6B shows that U266 cells were incubated alone or with STAT3iP (100 μM) for 1 h, and cellular localization of STAT3 was examined by immunocytochemistry (original magnification, 200X). FIG. 6C shows that U266 cells (2×10⁴ cell) were incubated alone or with 50 μM or 100 μM STAT3iP for indicated days and viable cells were counted using trypan blue cell viability test. Results are shown as mean (±s.d. indicated by error bar) of absorbance (570 nm) of triplicate cultures compared to the untreated control.

FIGS. 7A-B show& that IL-6 induces proliferation of human multiple myeloma cells and curcumin inhibits it. FIG. 7A shows that Human multiple myeloma cells U266, RPMI 8226, MM.1 and MM.1R were serum starved for 12 h and then cultured with the indicated concentrations of IL-6 for 48 h in a serum-free medium. FIG. 7B shows that U266, MM.1, and MM.1R cells were serum starved for 12 h and then cultured with the indicated concentrations of IL-6 in the absence or presence of curcumin (10 μM) for 48 h in a serum-free medium. Cell proliferation assays were performed as described below. Results are shown as mean (±s.d.) of percent [³H] thymidine incorporation or percent proliferation by MTT of triplicate cultures compared to the untreated control.

FIGS. 8A-C shows that TNF and LT can induce proliferation but does not induce STAT3 phosphorylation human multiple myeloma cells. FIG. 8A shows that MM.1S and MM.1R were serum starved for 12 hours and then cultured with the indicated concentrations of TNF and LT for 48 hours in a serum-free medium. The cell proliferation assay was performed as described below. Results are shown as mean (±s.d. indicated by error bar) of percent [³H] thymidine incorporation of triplicate cultures compared to the untreated control. FIG. 8B shows that MM.1S cells and FIG. 8C shows that MM.1R (2×10⁶ cells) were treated with 1 nM TNF or LT for indicated durations, and whole-cell extracts were prepared. Cells were treated with IL-6 for 10 minutes as a positive control for STAT3 phosphorylation. Thirty micrograms of whole cell extract was were resolved on 7.5% SDS-PAGE, electrotransferred to a nitrocellulose membrane, and probed for phosphorylated-STAT3 (upper panel), stripped, and reprobed for STAT3 (lower panel).

FIG. 9 shows the effect of curcumin on cell growth of dexamethasone-sensitive and dexamethasone-resistant MM cells. MM.1S and MM.1R cells were cultured with dexamethasone (1 μM) in the absence or presence of curcumin (10 μM) for 3 days and cell viability was checked by the MTT method. Results are shown as mean (±s.d. indicated by error bar) of percent cell growth of triplicate cultures compared to the untreated control.

FIG. 10 shows that IL-6 induces proliferation of human head and neck squamous cell carcinoma cells. Human head and neck squamous cell carcinoma cells MDA 1986LN, JMAR and MDA 686LN were serum starved for 12 hrs. These cells were then cultured with the indicated concentrations of IL-6 for 48 hrs in serum-free medium. Cell viability was determined by thymidine incorporation method.

FIGS. 11A-C show that STAT3 is constitutively phosphorylated in all head and neck squamous cell carcinoma cell lines except JMAR and that curcumin inhibits constitutive STAT3 phosphorylation in MDA 1986LN cells in a time- and dose-dependent manner. FIG. 11A shows the levels of p-STAT3 protein (upperpanel) and total STAT3 protein (lowerpanel) in the cells. TU167, MDA 1986LN, TU 686, MDA 686LN and JMAR cells (2×10⁶ cells) were lysed and whole cell extracts (30 μg) were resolved on 7.5% SDS-PAGE, electrotransferred to a nitrocellulose membrane and probed for phosphorylated-STAT3 (p-STAT3) and total STAT3 proteins. FIG. 11B shows the effect of treatment with curcumin for different durations on constitutive STAT3 phosphorylation in MDA 1986LN cells. FIG. 11C shows the effect of treatment with different concentrations of curcumin on constitutive STAT3 phosphorylation in MDA 1986LN cells. MDA 1986LN cells (2×10⁶ cells) were treated with curcumin (50 μM) for indicated durations or with indicated concentrations of curcumin for 1 h and levels of p-STAT3 (upper panels) and STAT3 (lower panels) were examined in whole cell extracts by Western blotting.

FIGS. 12A-B show that curcumin induces redistribution of STAT3 and inhibits STAT3 phosphorylation. FIG. 12A shows the effect of curcumin on STAT3 distribution in the cells. MDA 1986LN, MDA 686LN and JMAR cells were incubated with either media alone or with curcumin (50 μM) for 2 hrs and then analyzed by immunocytochemistry using Alexa fluorochrome for the distribution of STAT3 (original magnification ×200). FIG. 12B shows the effect of curcumin on STAT3 phosphorylation in the cells. The same procedure as mentioned above was performed on the same cells and the p-STAT3 analyzed by immunocytochemistry using horse radish peroxidase stain (original magnification ×200).

FIG. 13 shows that curcumin-induced inhibition of STAT phosphorylation is reversible. MDA 1986LN cells (2×10⁶ cells) were treated with curcumin (50 μM) for the indicated durations (upper panels) or treated for 1 h and washed twice with PBS to remove curcumin before re-suspending in fresh medium. Cells were removed at indicated times and lysed to prepare the whole cell extract (lower panels). The whole cell extracts (30 μg) were resolved on 7.5% SDS-PAGE, electrotransferred to a nitrocellulose membrane, probed for the p-STAT3 (upper panels) and striped and reprobed for STAT3 (lower panels).

FIGS. 14A-B show that IL-6 induces STAT3 phosphorylation in human head and neck squamous cell carcinoma JMAR cells and that curcumin inhibits the STAT3 phosphorylation in these cells. FIG. 14A shows the effect of IL-6 on STAT3 phosphorylation in JMAR cells. JMAR cells (2×10⁶ cells) were treated with IL-6 (10 ng/ml) for the indicated time and whole cell extracts were prepared. These extracts (30 μg) were resolved on 7.5% SDS-PAGE, electrotransferred to a nitrocellulose membrane and probed for p-STAT3 (upper panel) and stripped and re-probed for STAT3 (lower panel). FIG. 14B shows the effect of curcumin on IL-6 induced STAT3 phosphorylation in JMAR cells. JMAR cells were pretreated with curcumin (50 μM) for indicated durations and stimulated with IL-6 (10 ng/ml) for 10 mins and whole cell extract prepared and probed as mentioned earlier.

FIGS. 15A-C show that curcumin is a more potent inhibitor of STAT3 phosphorylation and cell proliferation than AG490 in MDA 1986LN cells. FIG. 15A shows the effect of treatment with different concentrations of AG490 on p-STAT3, STAT3 levels in these cells. MDA 1986LN cells (2×10⁶ cells) were treated with indicated concentrations of AG490 for 8 h and whole extracts prepared. These whole-cell extracts (30 μg) were resolved on 7.5% SDS-PAGE, electrotransferred to a nitrocellulose membrane, probed for p-STAT3 (upper panel), striped and reprobed for STAT3 (middle panel) and β-actin (lower panel). FIG. 15B shows the effect of treatment with AG490 for different durations on p-STAT3 and STAT3 levels in these cells. The cells were treated with AG490 (100 μM) for the indicated durations and whole cell extracts prepared and analyzed as mentioned earlier. FIG. 15C compares the effect of curcumin and AG490 on proliferation of MDA 1986LN cells by MTT assay.

### DETAILED DESCRIPTION OF THE INVENTION

The following abbreviations may be used herein. IL, interleukin; MM, multiple myeloma; STAT; signal transducers and activators of transcription; JAK, Janus kinase; NF-KB, nuclear factor-KB, TNF, tumor necrosis factor; LT, lymphotoxin; dex, dexamethasone; STAT3iP, STAT3 inhibitor peptide; IFN, interferon.

The present invention is directed to a method of treating a cancerous or pre-cancerous state in an individual in need of such treatment, comprising the step of administering a pharmacologically effective dose of a curcuminoid to said individual. Representative curcuminoid compounds may be selected from the group consisting of curcumin, demethoxycurcumin, and bisdemethoxycurcumin as well as any derivatives of these curcuminoid compounds or analogues thereof. A person having ordinary skill in this art would be readily able to determine the optimum dose and route of administration of a curcuminoid useful in the methods of the present invention. Generally, the curcuminoid is administered in a dose of from about 1 mg/kg to about 100 mg/kg. Although this method of the present invention may be useful to treat any cancerous or precancerous state, it is specifically contemplated that this method will be particularly useful when the individual is in a state characterized by constitutive activation of STAT3 expression. However, the method of the present invention can also be useful when the individual is in a state characterized by inducible activation of STAT3 expression. Representative examples of such states include multiple myeloma, head and neck cancers, hepatocellular carcinoma lymphoma and leukemia. Representative leukemias include chronic lymphocytic leukemia, acute myelogenous leukemia, large granular lymphocyte leukemia, erythroleukemia, polycythemia vera, adult T cell leukemia/lymphoma and acute lymphocytic leukemia. Representative lymphomas include EBV-related/Burkitt's, mycosis fungoides, cutaneous T-cell lymphoma, Hodgkin's disease, anaplastic lymphoma and B cell lymphoma. Represenative solid tumors which may be treated using the methods of the present invention include breast cancer, squamous cell carcinoma of the head and neck (SCCHN), renal cell carcinoma, melanoma, ovarian carcinoma, lung cancer, prostate carcinoma, pancreatic adenocarcinoma and brain tumor.

The present invention is also directed to a method of treating a cancerous or pre-cancerous state in an individual in need of such treatment, comprising the step of administering a pharmacologically effective dose of a curcuminoid and a chemotherapeutic agent to the individual. Representative chemotherapeutic agents include paclitaxel, 5FU, cisplatin, doxirubicin, dexamthasone, melphan, and gemcitabin.

The present invention is also directed to a method of reducing activated STAT3 expression in a cell, comprising the step of contacting the cell with pharmacologically effective dose of a curcuminoid. Representative useful curcuminoids include curcumin, demethoxycurcumin, and bisdemethoxycurcumin as well as any derivatives of these curcuminoid compounds. These curcuminoids may be administered in a dose of from about 1 mg/kg to about 100 mg/kg. In this method of the present invention, although it is desirable to inhibit both inducible and constitutively active STAT3 expression generally, it is most preferred that constitutively active STAT3 expression is inhibited. Although a person having ordinary skill in this art may find it useful to utilize this method of present invention to decrease activated STAT3 expression in a variety of diverse cell types, one preferred cell type is a multiple myeloma cell. Further preferred cell types include head and neck cancer cells, hepatocellular carcinoma cells, lymphoma cells or leukemia cells. Representative leukemias include chronic lymphocytic leukemia, acute myelogenous leukemia, large granular lymphocyte leukemia, erythroleukemia, polycythemia vera, adult T cell leukemia/lymphoma and acute lymphocytic leukemia. Representative lymphomas include EBV-related/Burkitt's, mycosis fungoides, cutaneous T-cell lymphoma, Hodgkin's disease, anaplastic lymphoma and B cell lymphoma. Representative solid tumors which may be treated using the methods of the present invention include breast cancer, scchn, renal cell carcinoma, melanoma, ovarian carcinoma, lung cancer, prostate carcinoma, pancreatic adenocarcinoma and brain tumor as well as administration to a post-surgical patient to prevent or inhibit re-occurence of the disease.

The present invention is also directed to a method of reducing activated STAT3 expression in a cell, comprising the step of contacting the cell with pharmacologically effective dose of a curcuminoid and a chemotherapeutic agent. Representative chemotherapeutic agents include paclitaxel, 5FU, cisplatin, doxirubicin, dexamthasone, melphan, and gemcitabin.

The following examples are given for the purpose of illustrating various embodiments of the invention and are not meant to limit the present invention in any fashion. The present examples, along with the methods, procedures, treatments, molecules, and specific compounds described herein are presently representative of preferred embodiments. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Materials

Human MM cell lines U266, RPMI 8226, and MM.1S were obtained from the American Type Culture Collection (Rockville, Md.). Cell lines U266 (ATCC#TIB-196) and RPMI 8226 (ATCC#CCL-155) are plasmacytomas of B cell origin. U266 is known to produce monoclonal antibodies and IL-6 (5, 25). RPMI 8226 produces only immunoglobulin light chains, and there is no evidence for heavy chain or IL-6 production. The MM.1 (also called MM.1S) cell line, established from the peripheral blood cells of a patient with IgA myeloma, secretes lambda light chain, is negative for the presence of EBV genome, and expresses leukocyte antigen DR, PCA-1, T9, and T10 antigens (26). MM.1R is a dexamethasone (dex)-resistant variant of MM.1 cells, also known as MM.1S (27), and was provided by Dr. Steven T. Rosen of Northwestern University Medical School (Chicago, Ill.). Human MM cell line OCl was provided by Dr. James Berenson from Cedar-Sinai Hospital (Los Angeles, Calif.).

The rabbit polyclonal antibodies to STAT1, STAT3, STAT5, and STAT6 and mouse monoclonal antibodies against phospho-STAT3 were obtained from Santa Cruz Biotechnology (Santa Cruz, Calif.). Goat anti-rabbit-horse radish peroxidase (HRP) conjugate was purchased from Bio-Rad Laboratories (Hercules, Calif.), goat anti-mouse-HRP from Transduction Laboratories (Lexington, Ky.), and goat anti-rabbit-Alexa 594 from Molecular Probes (Eugene, Oreg.). Hoechst 33342, and MTT were purchased from Sigma-Aldrich Chemicals (St. Louis, Mo.). Curcumin with a purity greater than 98%, was purchased from LKT laboratories, Inc. (St. Paul, Minn.), and was prepared as a 20 mM solution in dimethyl sulfoxide and then further diluted in cell culture medium. RPMI-1640, fetal bovine serum (FBS), 0.4% trypan blue vital stain, and antibiotic-antimycotic mixture were obtained from Life Technologies, Inc. (Grand Island, N.Y.). Protein A/G-Sepharose beads were obtained from Pierce (Rockford, Ill.), and [³H]thymidine from Amersham Biosciences (Piscataway, N.J.). Cell permeable STAT3 inhibitory peptide and AG490 were from Calbiochem (San Diego, Calif.). Bacteria-derived recombinant human IL-6 was provided by Sandoz Pharmaceutical (East Hanover, N.J.). Interferon (IFN)-α, was provided by Schering Plough Corporation. Bacteria-derived recombinant human TNF and LT were provided by Genentech Inc, (South San Francisco, Calif.).

### EXAMPLE 2

Cell Culture

All the human multiple myeloma cell lines were cultured in RPMI 1640 medium containing 1× antibiotic-antimycotic. U266, MM.1S, RPMI 8226 and MM.1R were cultured in 10% FBS, whereas cell line OCl was grown in Iscove's modified-Eagle's medium with 15% FBS. Cells were free of mycoplasma contamination as tested by Hoechst staining and by RT-PCR.

### EXAMPLE 3

Western Blot

For detection of STAT proteins, whole-cell extracts were prepared by lysing the curcumin-treated cells in lysis buffer (20 mM Tris, pH 7.4, 250 mM NaCl, 2 mM EDTA, pH 8.0, 0.1% Triton-X100, 0.01 mg/ml aprotinin, 0.005 mg/ml leupeptin, 0.4 mM PMSF, and 4 mM NaVO₄). Lysates were then spun at 14000 rpm for 10 min to remove insoluble material, and resolved on a 7.5% gel. After electrophoresis, the proteins were electrotransferred to a nitrocellulose membrane, blocked with 5% nonfat milk, and probed with anti-STAT antibodies (1:1000) overnight at 4° C. The blot was washed, exposed to HRP-conjugated secondary antibodies for 1 h, and finally examined by chemiluminescence (ECL, Amersham Pharmacia Biotech. Arlington Heights, Ill.).

### EXAMPLE 4

Immunocytochemistry for STAT3 Localization

Curcumin-treated multiple myeloma cells were plated on a glass slide by centrifugation using a Cytospin 4 (Thermoshendon, Pittsburg, Pa.), air-dried for 1 h at room temperature, and fixed with cold acetone. After a brief washing in PBS, slides were blocked with 5% normal goat serum for 1 h and then incubated with rabbit polyclonal anti-human STAT3 antibody (dilution, 1:100). After overnight incubation, the slides were washed and then incubated with goat anti-rabbit IgG-Alexa 594 (1:100) for 1 h and counter-stained for nuclei with Hoechst (50 ng/ml) for 5 min. Stained slides were mounted with mounting medium (Sigma Co.) and analyzed under an epifluorescence microscope (Labophot-2, Nikon, Tokyo, Japan). Pictures were captured using Photometrics Coolsnap CF color camera (Nikon, Lewisville, Tex.) and MetaMorph version 4.6.5 software (Universal Imaging Corp., Downingtown, Pa.).

### EXAMPLE 5

MTT Assay

The antiproliferative effects of curcumin against different multiple myeloma cell lines were determined by the MTT dye uptake method as described earlier (28). Briefly, the cells (5000/well) were incubated in triplicate in a 96-well plate in the presence or absence of indicated test samples in a final volume of 0.1 ml for 24 h at 37° C. Thereafter, 0.025 ml of MTT solution (5 mg/ml in PBS) was added to each well. After a 2-h incubation at 37° C., 0.1 ml of the extraction buffer (20% SDS, 50% dimethylformamide) was added and the extract incubated overnight at 37° C. for solubilization of formazan crystals. The OD at 570 nm was measured using a 96-well multiscanner autoreader (Dynatech MR 5000), with the extraction buffer serving as blank. Percent cell viability cell viability was calculated using the following formula:
Percent cell viability=(OD of the experiment samples/OD of the control)×100.

### EXAMPLE 6

Thymidine Incorporation Assay

The antiproliferative effects of curcumin were also monitored by the thymidine incorporation method. For this, 5000 cells in 100 μl medium were cultured in triplicate in 96-well plates in the presence or absence of curcumin for 24 h. Six hours before the completion of experiment, cells were pulse treated with 0.5 μCi [³H]thymidine, and the uptake of [³H]thymidine was monitored using a Matrix-9600 β-counter (Packard Instruments, Downers Grove, Ill.).

### EXAMPLE 7

Curcumin Inhibits Constitutive STAT3 Phosphorylation in Multiple Myeloma Cells

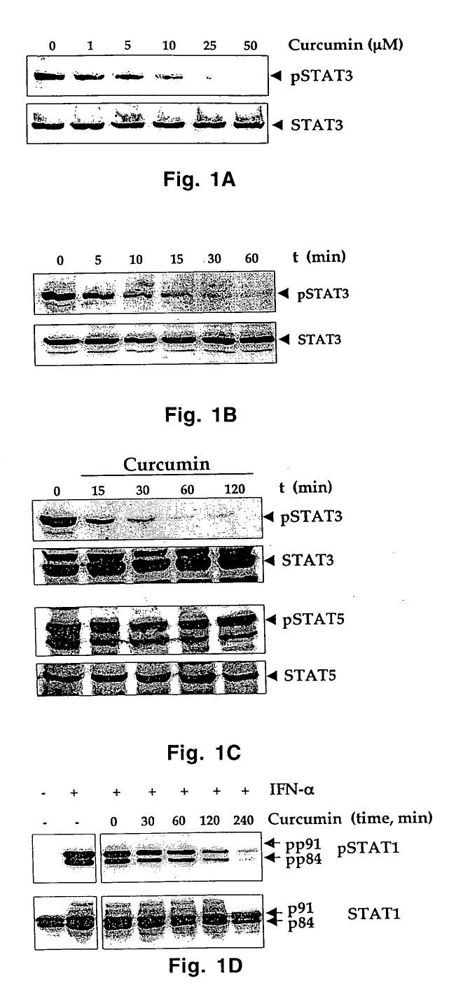

Whether curcumin inhibits the constitutive STAT3 phosphorylation in U266 was investigated. U266 cells were incubated either with different concentrations of curcumin for 1 h or with 50 μM curcumin for different times. Curcumin inhibited the constitutively active STAT3 in a dose-(FIG. 1A) and time-(FIG. 1B) dependent manner. Curcumin-induced inhibition could be observed as early as 10 min and with a concentration as low as 10 μM. Curcumin at 50 μM for 1 h completely inhibited STAT3 phosphorylation. Curcumin treatment did not alter the overall expression of STAT3 protein.

### EXAMPLE 8

Curcumin Does Not Inhibit Constitutive STAT5 Phosphorylation but Inhibits IFN-Inducible STAT1 Phosphorylation

Whether curcumin affects the phosphorylation of other STAT proteins in U266 cells was investigated. Besides STAT3, U266 cells expressed STAT5 (FIG. 1C). These cells also expressed constitutively phosphorylated STAT5. Hence, whether curcumin affected the constitutive phosphorylation of STAT5 was investigated. Under the conditions where curcumin completely inhibited the STAT3 phosphorylation, it neither altered the levels of constitutively phosphorylated STAT5, nor the expression of STAT5.

These results show that U266 cells expressed STAT1 but it was not phosphorylated (FIG. 1D). To determine the effect of curcumin on STAT1 phosphorylation, U266 cells were pretreated with IFN-a and then exposed to curcumin for different times. These results show that IFN-a induced STAT1 phosphorylation and curcumin suppressed the phosphorylation in a time-dependent manner (FIG. 1D).

### EXAMPLE 9

Curcumin Inhibits STAT3 Nuclear Translocation in Multiple Myeloma Cells

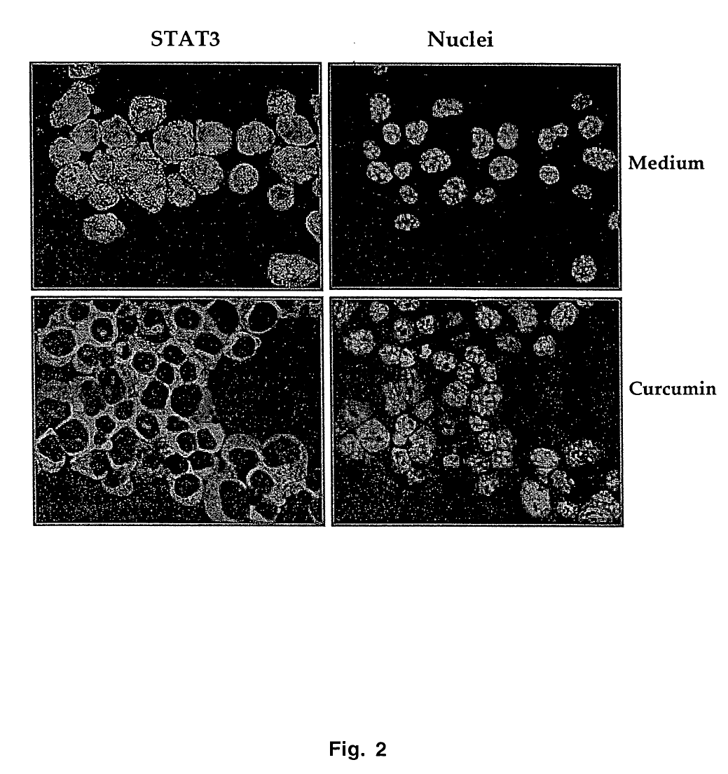

Under resting conditions, and in the nonphosphorylated state, STAT3 is retained in the cytoplasm. It translocates to the nucleus when phosphorylated (7). Phosphorylation induces STAT3 dimerization, thus permitting its translocation into the nucleus. To confirm that curcumin suppresses nuclear translocation of STAT3, curcumin-treated and untreated cells were cytospun on a glass slide, immunostained with antibody to STAT3, and then visualized by the Alexa-594 conjugated second antibody technique described above. FIG. 2 clearly demonstrates that curcumin prevented the translocation of the STAT3 to the nucleus in U266 cells, consistent with the curcumin-induced inhibition of STAT3 phosphorylation.

### EXAMPLE 10

Curcumin Inhibits IL-6-Inducible STAT3 Phosphorylation in Human Multiple Myeloma

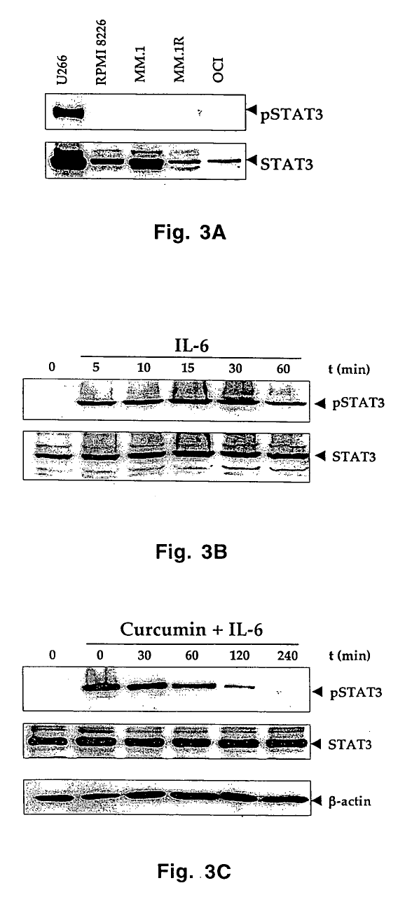

Since IL-6-induced signals are mediated through STAT3 phosphorylation, the status of STAT3 phosphorylation was examined. All multiple myeloma cell lines express STAT3 but only U266 expressed a constitutively phosphorylated STAT3 (FIG. 3A). These results are consistent with previous observations that only U266 constitutively secretes IL-6 (20).

Since IL-6 is a growth factor for multiple myeloma and induces STAT3 phosphorylation (5, 6, 15), whether curcumin could inhibit IL-6-induced STAT3 phosphorylation was examined. RPMI 8226 cells (which do not express constitutively phosphorylated STAT3) were treated with IL-6. IL-6 induced phosphorylation of STAT3 as early as 5 min and began to decline at 60 minutes (FIG. 3B). RPMI 8226 cells were then incubated with curcumin for different times and examined for IL-6-inducible STAT3 phosphorylation.

As seen in FIG. 3C, IL-6-induced STAT3 phosphorylation was blocked by curcumin in a time-dependent manner. Exposure of cells to curcumin for 4 hours was sufficient to completely suppress IL-6-induced STAT3 phosphorylation. Curcumin alone had no effect on STAT3 phosphorylation in these cells (data not shown).

### EXAMPLE 11

Curcumin-Induced Inhibition of STAT3 Phosphorylation is Reversible in Human Multiple Myeloma Cells

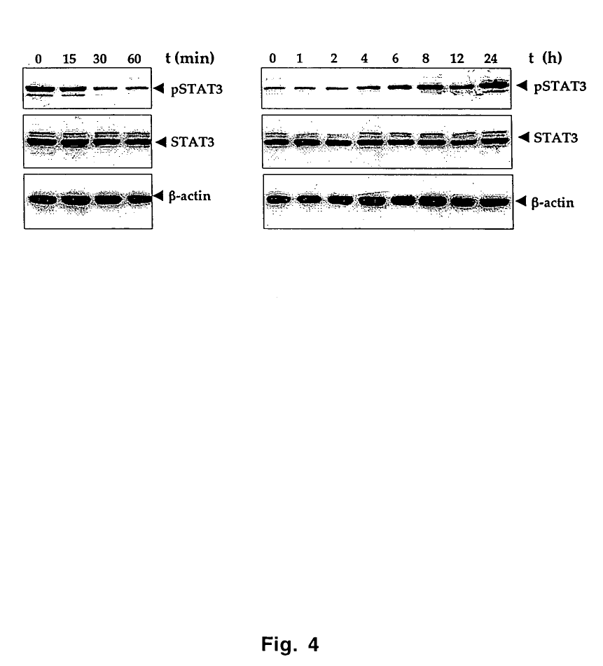

Whether curcumin-induced inhibition of STAT3 phosphorylation was reversible was further examined. U266 cells were first treated for 60 min with curcumin, and then the cells were washed twice with PBS to remove curcumin. The cells were then cultured in the fresh medium for various durations, and the levels of phosphorylated STAT3 were measured. Curcumin induced the suppression of STAT3 phosphorylation (FIG. 4, left panel), and the removal of curcumin resulted in gradual increase in phosphorylated STAT3 (FIG. 4, right panel). The reversal was complete by 24 hours and did not involve the changes in STAT3 levels.

### EXAMPLE 12

Curcumin is More Effective than AG490 in Inhibiting STAT3 Phosphorylation

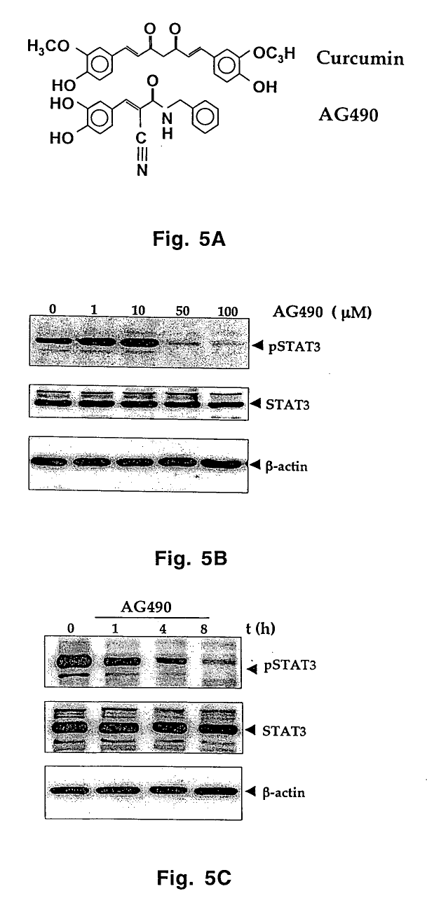

AG490 is a well-characterized inhibitor of STAT3 phosphorylation (29). It has certain structural features that are similar to curcumin's (see FIG. 5A). How the activity of AG490 compares with curcumin in inhibiting STAT3 phosphorylation was investigated.

A 100 μM dose of AG490 is needed to completely inhibit STAT3 phosphorylation. Next, the kinetics of inhibition by AG490 for STAT3 phosphorylation was first examined (FIG. 5B) and exposure of cells to 100 μM AG490 for 8 hours was needed to completely inhibit STAT3 phosphorylation (FIG. 5C). In comparison, treatment of cells with 50 μM curcumin for 60 minutes was sufficient to inhibit STAT3 phosphorylation (see FIGS. 1A and 1B).

### EXAMPLE 13

Cell Permeable STAT3 Inhibitor Peptide (STAT3iP) Inhibits Constitutive STAT3 Phosphorylation and U266 Cell Growth

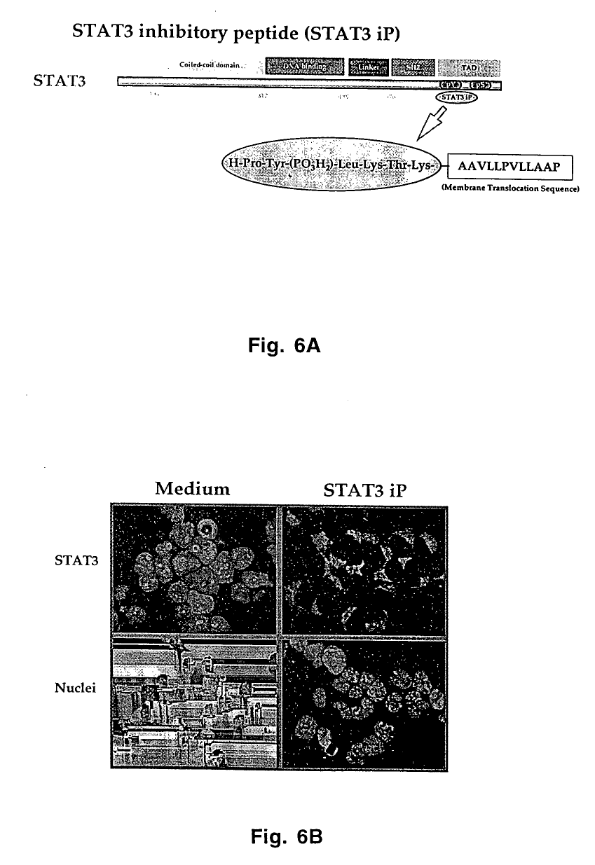

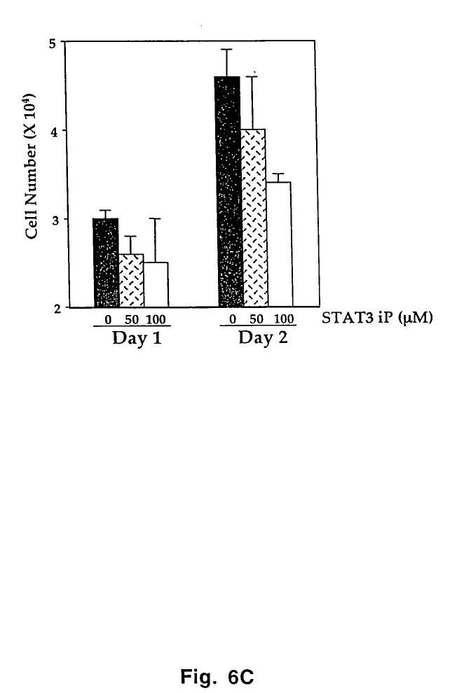

STAT3iP (FIG. 6A) is a cell-permeable analog of the STAT3-SH2 domain-binding phospho-peptide that acts as a highly selective, potent blocker of Stat3 activation (30). The effect of this peptide inhibitor of constitutive STAT3 phosphorylation was examined in U266 cells. Results in FIG. 6B show that 100 μM STAT3iP inhibits STAT3 phosphorylation completely within one hour.

### EXAMPLE 14

STAT3 Phosphorylation is Linked to Proliferation of MM Cells

Because STAT3 phosphorylation has been linked with the proliferation of MM cells, the effect of STAT3iP on the proliferation of U266 cells was examined. It was found that STAT3iP suppressed the U266 cells (FIG. 6C).

### EXAMPLE 15

IL-6 Induces Proliferation of Human Multiple Myeloma Cells Which is Inhibited by Curcumin

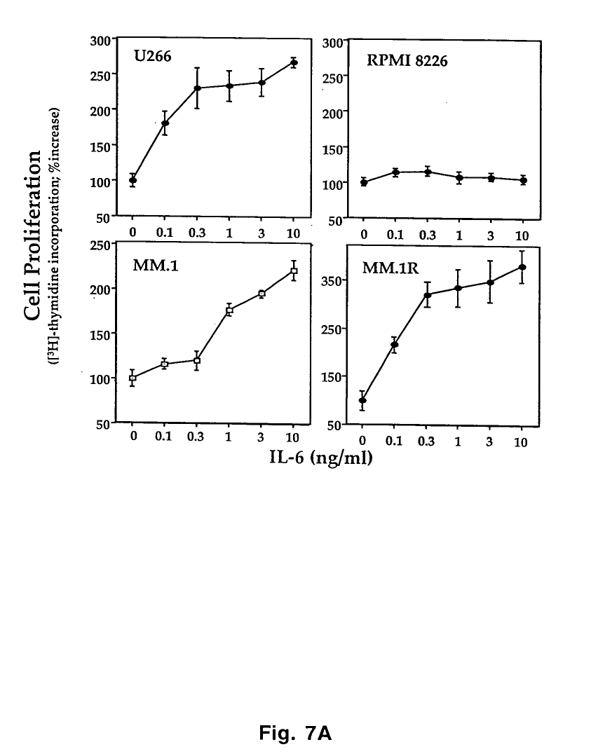

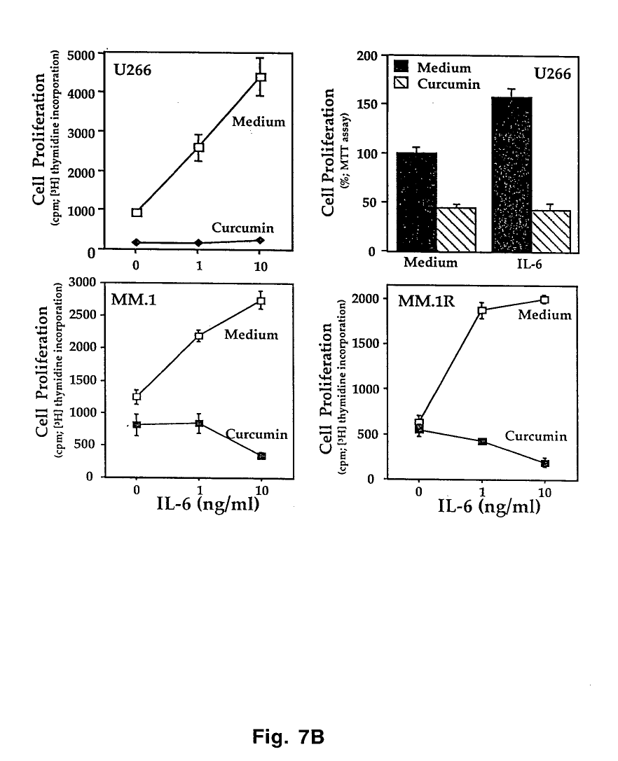

To reconfirm previous reports (5, 6, 15, 31) that IL-6 induces proliferation of MM cells, U266, RPMI 8226, MM.1 and MM.1R were serum-starved for 12 h and then cultured them in the absence or presence of different concentrations of IL-6 for 48 h. IL-6 induced proliferation of U266, MM.1 and MM.1R in a dose-dependent manner (FIG. 7A). RPMI 8226 cells, however, did not respond to IL-6 stimulation.

Whether curcumin suppresses the proliferative effects of IL-6 was determined. Both the thymidine incorporation (FIG. 7B, upper left panel) and MTT methods (FIG. 7B, upper right panel) showed that IL-6-induced proliferation of U266 cells was completely inhibited by curcumin. Similarly, curcumin also inhibited IL-6-induced cell proliferation in MM.1 and MM.1R cell lines (FIG. 7B, lower panels).

### EXAMPLE 16

TNF and LT Induce Proliferation of MM Cells Through STAT3-Independent Mechanism

Besides IL-6, TNF and lymphotoxin have been shown to be produced by multiple myeloma cells and are known to induce their proliferation (32-35). Furthermore, TNF has been shown to activate STAT3 signaling in different cell types (36, 37). Therefore whether TNF and lymphotoxin stimulation of multiple myeloma cells growth was mediated through STAT 3 phosphorylation was investigated.

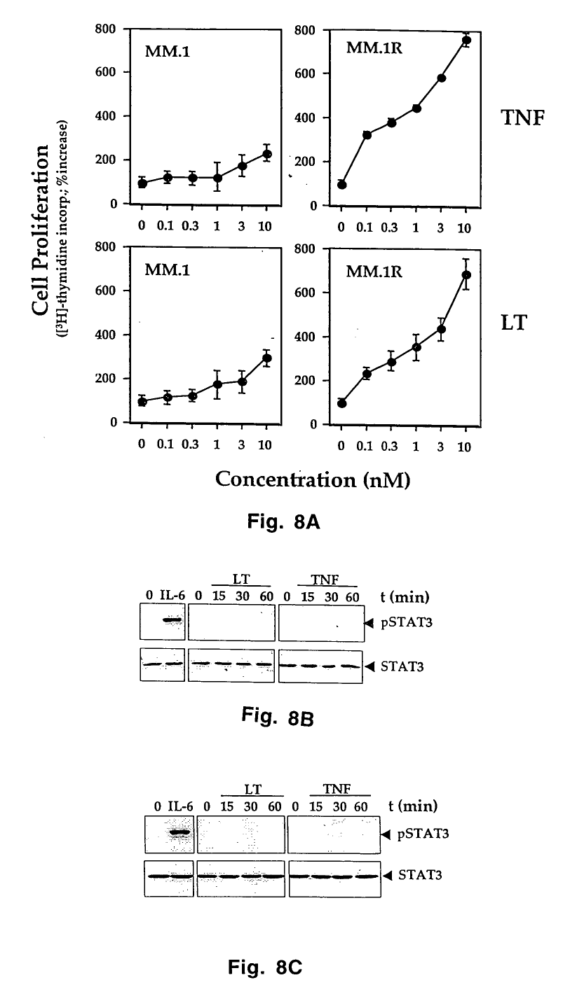

MM.1S and MM.1R cells were serum starved for 12 h and then treated with TNF or lymphotoxin in the serum free medium. The proliferative effects of TNF and lymphotoxin were more pronounced in dex-resistant MM.1R cells than in dex-sensitive MM.1S cells (FIG. 8A). When examined for STAT3 phosphorylation, IL-6 induced STAT3 phosphorylation in both cell lines but neither TNF nor lymphotoxin induced STAT3 phosphorylation in either cell line (FIG. 8B, 8C).

### EXAMPLE 17

Curcumin Inhibits the Growth of Dex-Resistant MM Cells

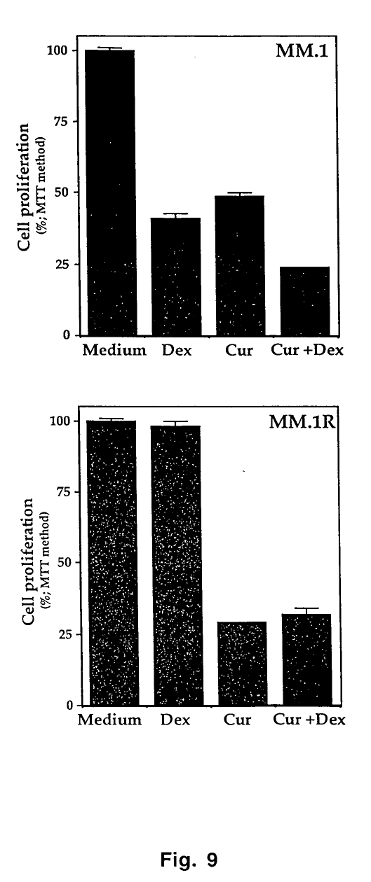

Multiple myeloma treated with dex gradually develops resistance to this drug. Although the mechanism of dex-resistance is not understood, it was found that curcumin inhibited the proliferation of dex-resistant and dex-sensitive multiple myeloma cells similarly (FIG. 9).

### EXAMPLE 18

IL-6 Induces Proliferation of Human HNSCC Cells

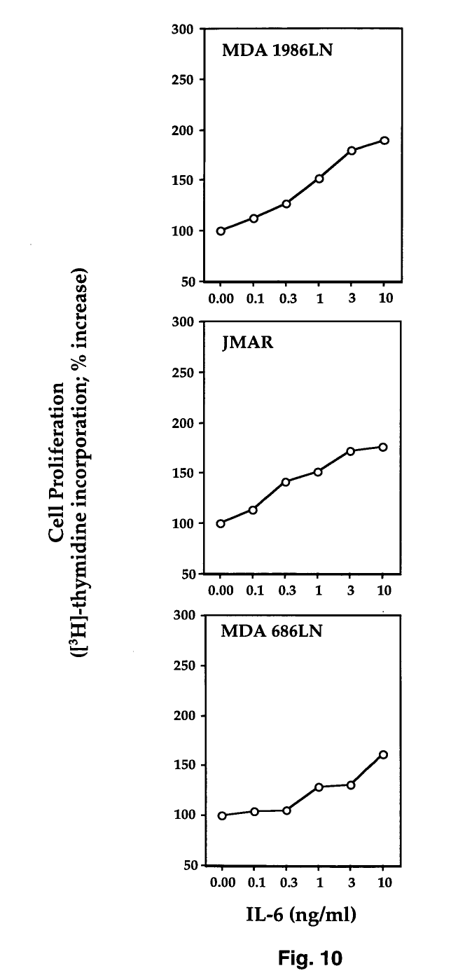

The present invention also investigated the effect of IL-6 on the proliferation of human HNSCC cells such as MDA 1986LN, JMAR and MDA 686LN by thymidine incorporation method. These cells were serum starved for 12 hrs and then cultured with different concentrations of IL-6 for 48 hrs in serum-free medium. IL-6 induced proliferation of MDA 1986LN, JMAR and MDA 686LN cells (FIG. 10).

### EXAMPLE 19

STAT3 is Constitutively Phosphorylated in HNSCC Cell Lines, Which is Inhibited by Curcumin.

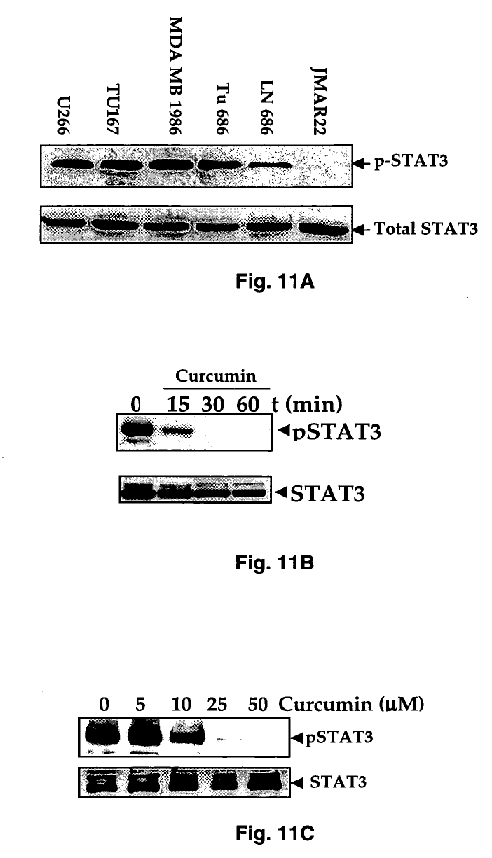

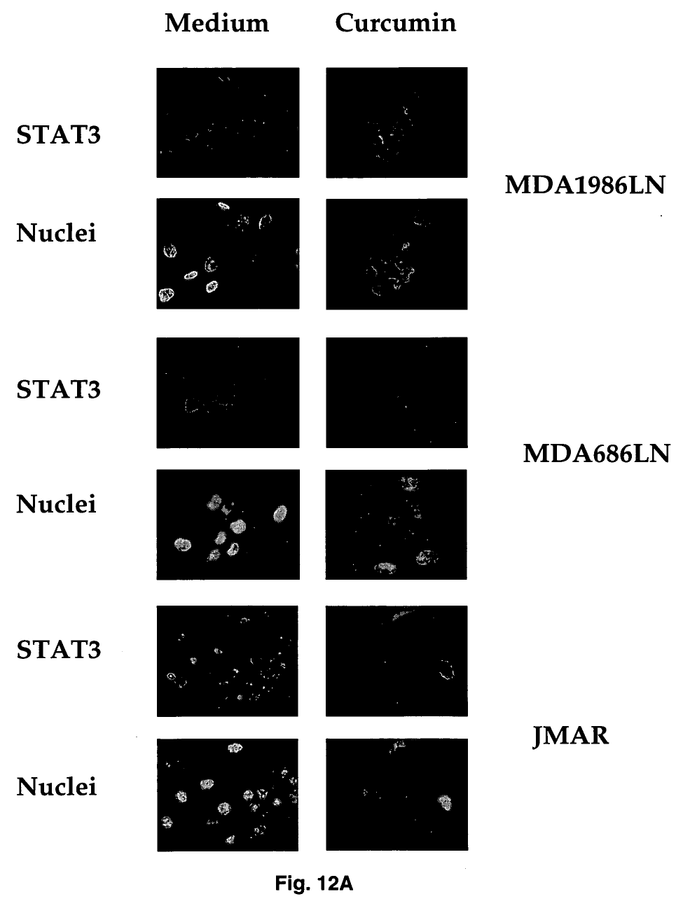

The present invention also investigated whether STAT3 was constitutively phosphorylated in head and neck squamous cell carcinoma cell lines such as TU 167, MDA 1986LN, TU 686, MDA 686LN and JMAR. Whole cell extracts of these cells were prepared and resolved on 7.5% SDS-PAGE and transferred on to nitrocellulose and probed for p-STAT3 and total STAT3. All the head and neck squamous cell carcinoma cell lines expressed STAT3 (FIG. 11A, lower panel). However, p-STAT3 was expressed in all the cells except JMAR cells (FIG. 11A, upper panel).

Since STAT3 was constitutively phosphorylated in MDA 1986LN cells, the ability of curcumin to inhibit the constitutive STAT3 phosphorylation was examined. MDA 1986LN cells were treated either with curcumin (50 μM) for different durations or with different concentrations of curcumin for 1 h and the p-STAT3 levels and STAT3 levels were determined. Curcumin inhibited the constitutively active STAT3 in a time-(FIG. 11B) and a dose-(FIG. 11C) manner. This curcumin-induced inhibition could be observed as early as 30 min and with a concentration as low as 25 μM. Curcumin at 50 μM for 1 hr inhibited the STAT3 phosphorylation completely. However, curcumin did not alter the overall expression of STAT3 protein.

### EXAMPLE 20

Curcumin Induces Redistribution and Inhibits Phosphorylation of STAT3 in HNSCC Cells

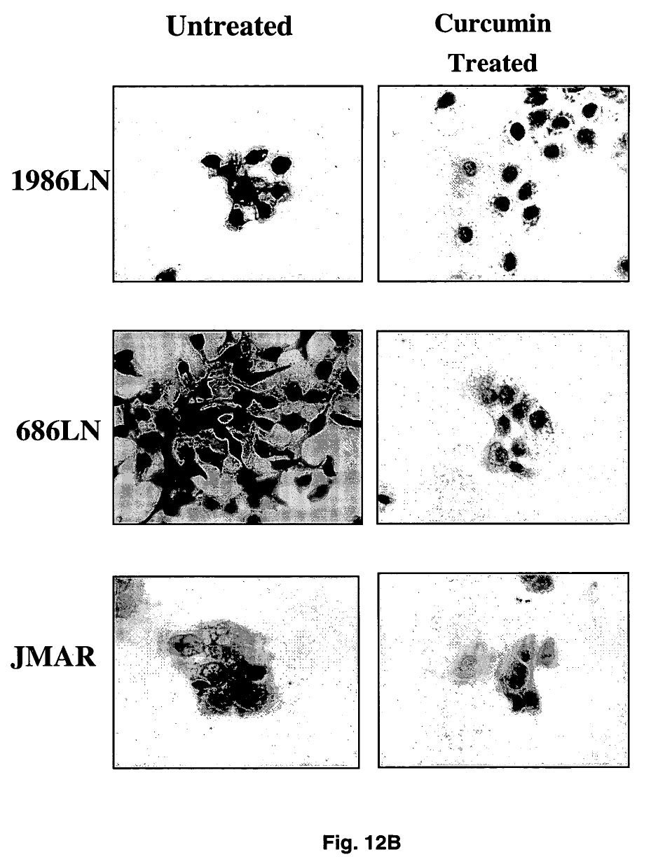

Since STAT3 retained in the cytoplasm under resting conditions and in the non-phosphorylated state translocates to nucleus when phosphorylated, the effect of curcumin on the distribution of STAT3 was examined. To accomplish this, curcumin-treated and untreated MDA 1986LN, MDA 686LN and JMAR cells were analyzed for distribution of STAT3 by immunocytochemistry using ALEXA fluorochrome. Curcumin prevented the translocation of STAT3 to the nucleus of these cells (FIG. 12A), which is consistent with the curcumin induced inhibition of STAT3 phosphorylation.

To further confirm that curcumin inhibited STAT3 phosphorylation, the same curcumin-treated and untreated cells were analyzed for phosphorylated STAT3 by immunocytochemistry using horseradish peroxidase stain. Curcumin inhibited the phosphorylation of STAT3 in MDA 1986LN and MDA 686LN cells (FIG. 12B, upper and middle panels). However, no inhibition of STAT3 phosphorylation was observed in curcumin-treated JMAR cells since they do not express constitutively active STAT3 protein (FIG. 12B, lower panel) (please confirm). These findings confirmed that curcumin inhibited STAT3 phosphorylation.

### EXAMPLE 21

Curcumin-Induced Inhibition of STAT3 Phosphorylation is Reversible in HNSCC Cells

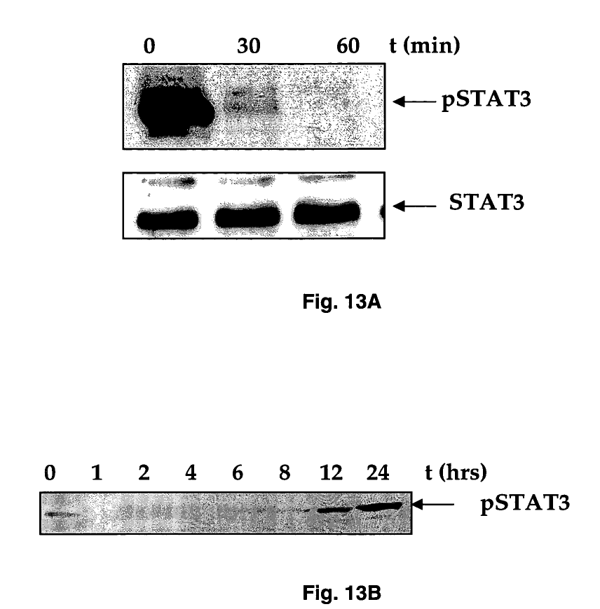

Since it was observed that curcumin inhibited the phosphorylation of STAT3 in head and neck squamous cell carcinoma cells, present investigation also investigated whether this inhibition was reversible. MDA 1986LN cells were treated with curcumin for indicated times or for 1 h and the cells were washed twice with PBS to remove curcumin. The cells were then cultured in media for various durations and the levels of phosphorylated STAT3 were determined. Curcumin induced inhibition of STAT3 phosphorylation (FIG. 13, upper panel) and the removal of curcumin resulted in gradual increase in phosphorylated STAT3 (FIG. 13, lower panel). However, the STAT3 levels were unaffected by curcumin treatment.

### EXAMPLE 22

IL-6 Induces STAT3 Phosphorylation in Human HNSCC JMAR Cells, Which is Inhibited by Curcumin

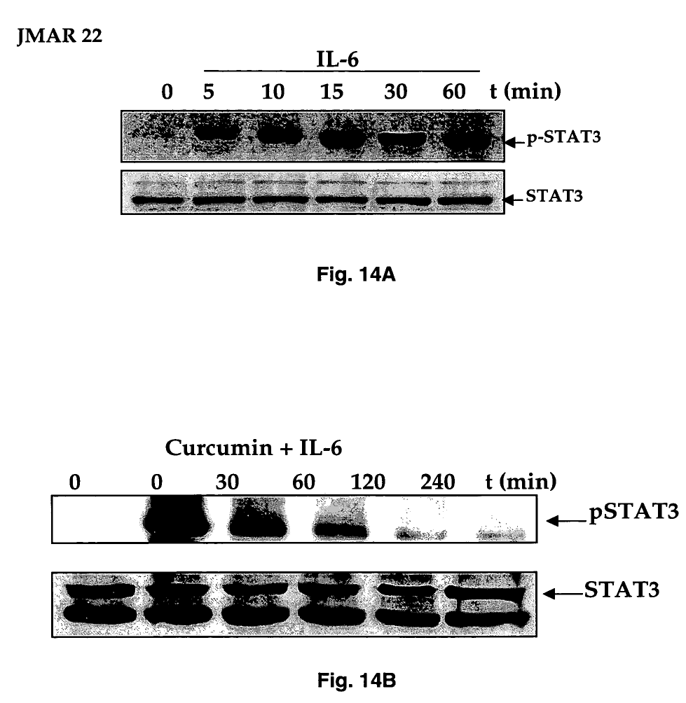

Since the present invention demonstrated that IL-6 induced proliferation of JMAR cells (FIG. 10, middle panel), the effect of IL-6 on the phosphorylation of STAT3 was investigated. JMAR cells were treated with IL-6 for different durations and the levels of p-STAT3 and STAT3 were examined. Treatment with IL-6 as early as 5 mins induced STAT3 phosphorylation in JMAR cells (FIG. 14A, upper panel). However, STAT3 levels were unaffected (FIG. 14A, lower panel).

Further, since IL-6 induced STAT3 phosphorylation in JMAR cells, the ability of curcumin to inhibit this STAT3 phosphorylation was also examined. JMAR cells were pretreated with curcumin for different durations and stimulated with IL-6 for 10 minutes. Both the status of p-STAT3 and STAT3 were determined in the whole cell extracts of these cells. Treatment with curcumin for as early as 30 minutes led to decline in the phosphorylation of STAT3 which declined further with increased exposure to curcumin. Exposure to curcumin for 2 hours inhibited STAT3 phosphorylation in JMAR cells (FIG. 14B, upper panel). The STAT3 levels were unaffected by this treatment (FIG. 14B, lower panel).

### EXAMPLE 23

Curcumin is a More Potent Inhibitor of STAT3 Phosphorylation and Cell Proliferation than AG490 in MDA 1986LN Cells

The ability of curcumin to inhibit STAT3 phosphorylation and cell proliferation was then compared with AG490, a well-characterized inhibitor of STAT3 phosphorylation (29).

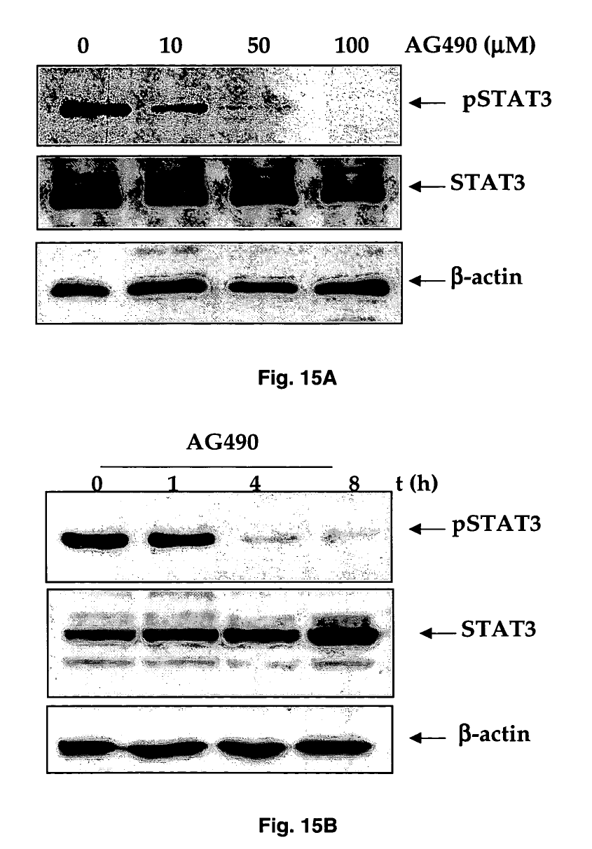

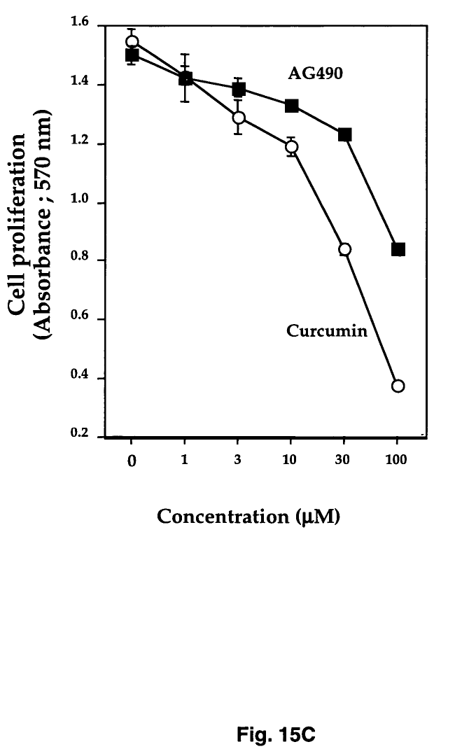

The concentration and the exposure time for AG490 to inhibit STAT3 phosphorylation in MDA 1986LN cells were determined. These cells were incubated with either different concentrations of AG490 or with 100 μM of AG490 for different durations and the levels of p-STAT3 and STAT3 were assessed. Exposure of cells to 100 μM of AG490 for 8 hrs completely inhibited STAT3 phosphorylation (FIG. 15A and 15B). In comparison, curcumin at 50 μM for 1 h was able to completely inhibit STAT3 phosphorylation in these cells (FIGS. 11B and 11C).

Further, the proliferation of MDA 1986LN cells when treated with curcumin was compared with AG490 treated cells by performing MTT assay. Curcumin inhibited proliferation of MDA 1986LN more efficiently that AG490 (FIG. 15C).

Because STAT3 phosphorylation plays a critical role in transformation and proliferation of tumor cells, the effect of curcumin on STAT3 phosphorylation in human multiple myeloma cells and in human HNSCC was investigated. It was found that curcumin abrogated both constitutive and IL-6 induced phosphorylation of STAT3 in certain multiple myeloma cells, but had no effect on STAT5 phosphorylation. Curcumin-induced inhibition of STAT3 phosphorylation was reversible. Curcumin was found to be a more rapid and more potent inhibitor of STAT3 phosphorylation than AG490. Curcumin suppressed the proliferative effects of IL-6. TNF-α and LT also induced the proliferation of multiple myeloma cells but through a mechanism independent of STAT3 phosphorylation. Multiple myeloma cells were found to be sensitive to curcumin regardless of whether they were resistant (MM.1R) or sensitive (MM.1S) to dexamethasone.

Only one of five multiple myeloma cell lines (U266) tested expressed constitutively active STAT3. Others have also shown that STAT3 is constitutively active in U266 cells (15) and IL-6 induces proliferation of these cells (15, 31, 38). Catlett-Falcone et al. showed that almost one 8 of 24 multiple myeloma patients showed constitutively active STAT3 (15). Constitutive active STAT3 has been found to be oncogenic and to transform wide variety of cells including breast, lymphoid, and myeloid cells (11-14). Constitutively active STAT3 has been implicated in the induction of resistance to apoptosis(15), possibly through the expression of Bcl-x_{L} and cyclinDi (23, 24).

These results show that curcumin completely eliminated the constitutively phosphorylated form of STAT3. Curcumin also abolished the IL-6-induced STAT3 phosphorylation in multiple myeloma cells that do not express constitutive STAT3. The effect of curcumin was specific in that it did not affect the phosphorylation state of STAT5 in multiple myeloma cells. Previously, it was shown that curcumin downregulates the expression of cyclinD1 and Bcl-x_{L} in multiple myeloma cells (20-22). These two genes are known to be regulated by both STAT3 and NF-κB (23, 24) and the downregulation of NF-κB by curcumin in multiple myeloma cells has been reported (20). Thus it is possible that curcumin downregulates the expression of cyclinD1 and Bcl-x_{L} through downregulation of both NF-κB and STAT3 activation. It was found that suppression of STAT3 phosphorylation was reversible, returning to control values within 24 h.

These results indicate that exogenous IL-6 induced the proliferation of U266 and MM.1R cells but not RPMI 8226 cells. Even though U266 cells express constitutively active STAT3 and secrete IL-6, their optimum growth still appears to be dependent on exogenous IL-6. In contrast, RPMI 8226 cells, which have no constitutively active STAT3, required IL-6 exposure for activation of STAT3. The activation of this STAT3, however, was not sufficient for the proliferation of RPMI 8226 cells. TNF and LT also induced proliferation of multiple myeloma cells but this was independent of STAT3 phosphorylation. These results further indicate that curcumin blocked IL-6 induced proliferation of multiple myeloma cells.

AG490, probably the best known inhibitor of STAT3 phosphorylation (29), was a less potent inhibitor of STAT3 phosphorylation than curcumin. A longer exposure (12 h vs 30 min) and higher dose (100 μM vs 10 μM) of AG490 was needed to suppress STAT3 phosphorylation. Similarly, while exposure of cells to 100 μM curcumin for 24 hours completely suppressed the proliferation of multiple myeloma cells, the same dose and duration of exposure to AG490 had no effect (data not shown).

AG490 is considered an inhibitor of JAK2 (29), a kinase that phosphorylates STAT3. Several other kinases have been implicated in the phosphorylation of STAT3, including members of the src family (hck, src), Erb2, ALK, PKC-δ, c-fes, and EGFR (42-55). Whether any of these kinases are active in multiple myeloma cells and which of these kinases are activated by IL-6 is not fully known. AG490 is known to inhibit MAPK pathway (56) as well as JAK2 pathway. Among the kinases known to phosphorylate STAT3, which is inhibited by curcumin is not known. There are reports that curcumin can inhibit JAK2 (39, 40), Src (57) and Erb2 (58) and EGFR (59), and so inhibition of any of these could explain the inhibitory effects of curcumin on STAT3 phosphorylation. Whether suppression of cell proliferation by curcumin is only due to inhibition of the nuclear translocation of STAT3, requires further investigation.

Recently, STATiP has been developed as a highly selective and potent inhibitor of STAT3 activation (30). STATiP suppresses constitutive STAT3 activation mediated by Src. These results indicate that STAT3iP expresses a similar STAT3 inhibitory activity to curcumin. Both agents block STAT3 phosphorylation within one hour. In addition, STAT3iP also suppressed the growth of multiple myeloma cells. STAT3iP was less effective in its growth suppressive effect compared to curcumin, which indicates the suppression of other transcription factors, which promote cell viability and proliferation. The essential role of NF-κB in survival and proliferation of multiple myeloma cells was recently described (20). Since curcumin can effectively inhibit activation of both STAT3 and NF-κB, it is expected that curcumin should suppress cell proliferation more efficiently than specific inhibitors of either transcription factor alone.

In addition to multiple myeloma cells, it was found that IL-6 induced proliferation of all the three human HNSCC cells that were tested (MDA 1986LN, JMAR and MDA 686LN). It was also found that STAT3 was constitutively phosphorylated in all HNSCC cell lines that were tested except JMAR cells. Curcumin inhibited constitutive STAT3 phosphorylation in MDA 1986LN cells in a time and dose dependent manner. Additionally, it was found that curcumin induced redistribution of STAT3 and inhibited STAT3 phosphorylation in these cells. Further, it was also found that the curcumin-induced inhibition of STAT3 phosphorylation was reversible. Since IL-6 induced proliferation of JMAR cells, the ability of IL-6 to induce STAT3 phosphorylation was also examined. It was found that IL-6 induced STAT3 phosphorylation and curcumin inhibited the IL-6 induced p-STAT3 in these cells. Therefore, curcumin was able to inhibit both constitutive and inducible phosphorylation of STAT3. Additionally, it was also found that curcumin was a more potent inhibitor of both STAT3 phosphorylation and cell proliferation than AG490.

Because there is considerable evidence that STAT3 is involved in the transformation of cells, of STAT3's activity as a transcriptional activator is a prime anticancer target. First, all Src-transformed cell lines have persistently activated STAT3 and dominant-negative STAT3 blocks transformation (60, 61). Dominant-negative STAT3 has also been shown to induce apoptosis in cells with constitutively active STAT3 (15). Second, STAT3-C, a constitutively active mutant dimerized by cycteine-cysteine bridges instead of pTyr-SH2 interaction, can transform cultured cells so that they form tumors when injected into mice (62). Indeed, STAT3 functions in normal lymphocyte development to resist apoptosis (63, 64). Third, besides multiple myeloma, head and neck cancers (65), hepatocellular carcinoma (66), lymphomas and leukemia (67) have constitutively active STAT3. Because there is no reported mutation in STAT3 that results in persistent activation, the only putative mechanism to account for the constitutive activity of STAT3 is dysregulation of signaling molecules or mutation or deletions in the protein that negatively regulate STAT3 (e.g; PIAS3 or SOCS) (66). For instance SOCS-1, a negative regulator of cytokine signaling, is frequently silenced by methylation (68). Thus constitutively active STAT3 can contribute to oncogenesis by protecting cancer cells from apoptosis. This implies that suppression of STAT3 activation by agents such as curcumin as described here could facilitate apoptosis.

These results also indicate that curcumin can overcome dex resistance of multiple myeloma cells. Thus in conclusion, the ability to suppress STAT3 phosphorylation, inhibit IL-6 signaling, downregulate the expression of IL-6, cyclin D1 and bcl-x_{L} (20), inhibit proliferation of multiple myeloma cells, and overcome drug resistance, combined with its well established pharmacological safety (69-73), suggest that curcumin should be tested in multiple myeloma patients. Additionally, the ability of curcumin to inhibit constitutive as well as inducible STAT3 phosphorylation as well as inhibit proliferation of human HNSCC cells combined with its well-established pharmacological safety (69-73), suggests that curcumin should be tested in human HNSCC patients.

The following references were cited herein:
1. Hallek, et al., 1998. Blood 91:3.
2. Forum, T. U. M. 2001. Br J Haematol 115:522.
3. Report. 1992. 1973-1989 Annual Cancer Statistics Review. National Cancer Institute, Bethesda.
4. Feinman, et al., 1999. Blood 93:3044.
5. Kawano, et al. 1988. Nature 332:83.
6. Klein, et al., 1995. Blood 85:863.
7. Darnell et al., 1997. Science 277:1630.
8. Taga, T., and T. Kishimoto. 1997. Annu Rev Immunol 15:797.
9. Bromberg, et al., 1998. Mol Cell Biol 18:2553.
10. Turkson, et al., 1998. Mol Cell Biol 18:2545.
11. Garcia, R., and R. Jove. 1998. Activation of STAT transcription factors in oncogenic tyrosine kinase signaling. J Biomed Sci 5:79.
12. Garcia, et al., 1997. Cell Growth Differ 8:1267.
13. Weber-Nordt, et al., 1996. Blood 88:809.
14. Takemoto, etal., 1997. Proc Natl Acad SciUSA 94:13897.
15. Catlett-Falcone, 1999. Immunity 10:105.
16. Tu, et al., 1998. Cancer Res 58:256.
17. Grad, et al., 2000. Curr Opin Oncol 12:543.
18. Pettersson, et al., 1992. Blood 79:495.
19. Tu, et al., 1996. Blood 88:1805.
20. Bharti, et al., 2003. Blood 101:1053.
21. Han, et al., 1999. Clin Immunol 93:152.
22. Mukhopadhyay, et al., Oncogene In press.
23. Fujio, et al., 1997. J Clin Invest 99:2898.
24. Sinibaldi, et al., 2000. Oncogene 19:5419.
25. Nilsson, K et al., 1970. Clin Exp Immunol 7:477.
26. Goldman-Leikin, et al., 1989. J Lab Clin Med 113:335.
27. Moalli, et al., 1992. Blood 79:213.
28. Manna, et al., 1998. J Biol Chem 273:13245.
29. Meydan, et al., 1996. Nature 379:645.
30. Turkson, et al., 2001. J Biol Chem 276:45443.
31. Schwab, et al., 1991. Blood 77:587.
32. Borset, M., A. Waage, O. L. Brekke, and E. Helseth. 1994. Eur J Haematol 53:31.
33. Hata, H., H. Matsuzaki, and K. Takatsuki. 1990. Acta Haematol 83:133.
34. Bataille, et al., 1989. Cancer 63:877.
35. Garrett, et al., 1987. N Engl J Med 317:526.
36. Miscia et al., 2002. Cell Growth Differ 13:13.
37. Guo, et al., 1998. J Immunol 160:2742.
38. Keller, E. T., and W. B. Ershler. 1995. J Immunol 154:4091.
39. Li, et al., 2001. J Immunol 166:3491.
40. Natarajan, C., and J. J. Bright. 2002. J Immunol 168:6506.
41. Kopantzev, Y., M. Heller, N. Swaminathan, and S. Rudikoff. 2002. Oncogene 21:6791.
42. Schreiner, et al., 2002. J Biol Chem 277:45680.
43. Ren, Z., and T. S. Schaefer. 2002. J Biol Chem 277:38486.
44. Zamo, et al., v2002. Oncogene 21:1038.
45. Garcia, et al., 2001. Oncogene 20:2499.
46. Zhang, et al., 2000. J Biol Chem 275:24935.
47. Zong, et al., 2000. J Biol Chem 275:15099.
48. Wang, et al., 2000. Oncogene 19:2075.
49. Jain, et al., 1999. J Biol Chem 274:24392.
50. Sellers, et al., 1999. J Biol Chem 274:16423.
51. Liu, et al., 1998. J Biomed Sci 5:446.
52. Park, et al., 1998. Cancer Lett 129:29.
53. Chaturvedi, et al., 1998. Oncogene 16:1749.
54. Park, et al., 1996. Proc Natl Acad Sci U S A 93:13704.
55. Nelson, et al., 1998. J Biol Chem 273:7072.
56. De Vos, et al., 2000. Br J Haematol 109:823.
57. Reddy, S., and B. B. Aggarwal. 1994. FEBS Lett 341:19.
58. Hong, et al., 1999. Clin Cancer Res 5:1884.
59. Korutla, et al., 1994. Biochim Biophys Acta 1224:597.
60. Yu, et al., 1995. Science 269:81.
61. Bowman, et al., 2000. STATs in oncogenesis. Oncogene 19:2474.
62. Bromberg, et al., 1999. Stat3 as an oncogene. Cell 98:295.
63. Takeda, et al., 1998 J Immunol 161:4652.
64. Shen, et al., 2001 Proc Natl Acad Sci U S A 98:1543.
65. Song, J. I., and J. R. Grandis. 2000. STAT signaling in head and neck cancer. Oncogene 19:2489.
66. Yoshikawa, et al., 2001 Nat Genet 28:29.
67. Zhang, et al., 2002. J Immunol 168:466.
68. Galm, et al., 2002. Blood.
69. Lal, et al., 2000. Phytother Res 14:443.
70. Lal, et al., 1999. Phytother Res 13:318.
71. Plummer, et al., 2001. Cancer Epidemiol Biomarkers Prev 10:1295.
72. Cheng, et al., 2001. Anticancer Res 21:2895.
73. Sharma, et al. 2001 Clin Cancer Res 7:1894.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

1. A method of treating a cancerous or pre-cancerous state in an individual in need of such treatment, comprising the step of administering a pharmacologically effective dose of a curcuminoid to said individual.

2. The method of claim 1, wherein said curcuminoid is selected from the group consisting of curcumin, demethoxycurcumin, and bisdemethoxycurcumin or analogues thereof.

3. The method of claim 2, wherein said curcuminoid is administered in a dose of from about 1 mg/kg to about 100 mg/kg.

4. The method of claim 1, wherein said state is characterized by constitutive activation STAT3 expression.

5. The method of claim 1, wherein said state is characterized by inducible activation of STAT3 expression.

6. The method of claim 1, wherein said state is selected from the group consisting of multiple myeloma, head and neck cancers, hepatocellular carcinoma, lymphomas and leukemia.

7. The method of claim 6, wherein said leukemia is selected from the group consisting of chronic lymphocytic leukemia, acute myelogenous leukemia, large granular lymphocyte leukemia, erythroleukemia, polycythemia vera, adult T cell leukemia/lymphoma and acute lymphocytic leukemia.

8. The method of claim 6, wherein said lymphoma is selected from the group consisting of EBV-related/Burkitt's, mycosis fungoides, cutaneous T-cell lymphoma, Hodgkin's disease, anaplastic lymphoma and B cell lymphoma.

9. The method of claim 1, wherein said state is selected from the group consisting of breast cancer, scchn, renal cell carcinoma, melanoma, ovarian carcinoma, lung cancer, prostate carcinoma, pancreatic adenocarcinoma and brain tumor.

10. The method of claim 1, further comprising the step of administering to said individual a chemotherapeutic agent.

11. The method of claim 10, wherein said chemotherapeutic agent is selected from the group consisting of paclitaxel, 5FU, cisplatin, doxorubicin, dexamthasone, melphan, and gemcitabin.

12. A method of reducing activated STAT3 expression in a cell, comprising the step of contacting said cell with pharmacologically effective dose of a curcuminoid.

13. The method of claim 12, wherein said curcuminoid is selected from the group consisting of curcumin, demethoxycurcumin, and bisdemethoxycurcumin or analogues thereof.

14. The method of claim 12, wherein said curcuminoid is administered in a dose of from about 1 mg/kg to about 100 mg/kg.

15. The method of claim 12, wherein said activated STAT3 expression is constitutive.

16. The method of claim 12, wherein said activated STAT3 expression is inducible.

17. The method of claim 12, wherein said cell is a multiple myeloma cell.

18. The method of claim 12, wherein said cell is a head and neck cancer cell, a hepatocellular carcinoma cell, a lymphoma cell or a leukemia cell.

19. The method of claim 18, wherein said leukemia is selected from the group consisting of chronic lymphocytic leukemia, acute myelogenous leukemia, large granular lymphocyte leukemia, erythroleukemia, polycythemia vera, adult T cell leukemia/lymphoma and acute lymphocytic leukemia.

20. The method of claim 18, wherein said lymphoma is selected from the group consisting of EBV-related/Burkitt's, mycosis fungoides, cutaneous T-cell lymphoma, Hodgkin's disease, anaplastic lymphoma and B cell lymphoma.

21. The method of claim 12, wherein said cell is selected from the group consisting of breast cancer, scchn, renal cell carcinoma, melanoma, ovarian carcinoma, lung cancer, prostate carcinoma, pancreatic adenocarcinoma and brain tumor.

22. The method of claim 12, further comprising the step of contacting said cell with a chemotherapeutic agent.

23. The method of claim 22, wherein said chemotherapeutic agent is selected from the group consisting of paclitaxel, 5FU, cisplatin, doxorubicin, dexamthasone, melphan, and gemcitabin.
